(19) United States
(12) Patent Application Publication
(10) Pub. No.: US 2010/0113926 A1
(43) Pub. Date: May 6, 2010

(54) SYSTEM AND METHOD FOR CLUTTER FILTER PROCESSING FOR IMPROVED ADAPTIVE BEAMFORMING

(75) Inventors: Kenneth Wayne Rigby, Clifton Park, NY (US); Kjell Kristoffersen, Oslo (NO); Morten Lennart Haugen, Toensberg (NO); Dongxing He, Menomonee Falls, WI (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/262,430

(22) Filed: Oct. 31, 2008

(51) Int. Cl. A61B 8/00

(52) U.S. Cl. 600/437

(57) ABSTRACT

A method for clutter filter processing is provided. The method provides for extracting a blood component from an element signal produced by an element to obtain a filtered element signal, and extracting a blood component from a beamsum signal to obtain a filtered beamsum signal, calculating a time delay estimate between the filtered element signal and the filtered beamsum signal, or between the filtered element signal and the beam sum signal, or between the element signal and the filtered beamsum signal and applying the time delay estimate to correct transmit and receive beamforming time delays for the element. Systems and apparatus that afford functionality of the type defined by this method may be provided by the present technique.

### BACKGROUND

Embodiments of the invention relate generally to imaging systems and more specifically to a method and system for improving image resolution and contrast in an ultrasound imaging system.

Typically, ultrasound systems comprise an array of transducer elements used for transmitting a set of waveforms into an imaging subject and for receiving a set of reflected ultrasound signals. Each waveform is emitted with a relative time delay chosen to focus the net transmitted waveform in a desired direction and depth and with a desired shape. Similarly each received signal is individually delayed to maximize the response of the system to reflected energy for a desired direction and depth and with a desired shape. The delayed receive signals are summed and processed to create and display an image of the imaging subject.

The transmit and receive time delays, known collectively as beamforming time delays, are typically calculated based on the assumption that sound propagates through the body with a known, constant speed. However, it is observed that different tissue types (e.g., muscle, fat, cartilage, etc.) vary significantly in their corresponding sound speed. For example, cardiac imaging is often performed by placing the transducer between two of the subject's ribs. This acoustic window contains complicated layers of fat and intercostal muscles, so that the assumption of uniform sound speed propagation may not be valid. When this assumption fails, the transmit and receive focusing is degraded and there will be a resulting loss of image resolution and contrast.

Conventional methods of estimating time delay corrections for ultrasound imaging are unlikely to produce reliable time delay estimates when imaging the heart. These methods either directly or indirectly estimate time delay differences between individual transducer element signals, or between transducer element signals and the sum of these element signals, or between various sub-groups transducer element signals. Nearly all time-delay estimation methods explicitly or implicitly weight the contributions of strongly reflecting scatterers more heavily than weakly reflecting scatterers. However, scattering of ultrasound from the blood pool in the interior of the heart is much weaker than scattering from the heart wall. Thus, when imaging the interior of the heart, it is quite likely that the reflected ultrasound signal may be dominated by strongly reflecting heart wall tissue in the sidelobes of the ultrasound beam and not by the weakly reflecting blood in the mainlobe of the ultrasound beam. Hence, current time-delay estimation algorithms are likely to produce time-delay corrections which erroneously steer the corrected beams toward the strongly reflecting heart wall tissue.

A similar problem arises when imaging any weakly scattering object which is surrounded by strong scatterers, such as blood vessels and the gall bladder. Rescaling the amplitude of the signals that are compared does not solve this problem. For example, consider the limit in which only the sign of the signals is used in the time-delay estimation. When both blood and tissue contribute to a particular sample, the sign of that sample will still tend to be dominated by the sign of the much larger tissue contribution. Rescaling the amplitude helps only when some of the samples used in the time-delay estimation are dominated by blood.

The display of blood flow information in the ultrasound image, commonly known as color flow or color power, is a widely used medical diagnostic tool. At deeper imaging depths, however, it is often the case that conventional color flow processing is unable to extract a usable blood velocity or power signal, especially when imaging subjects in which large beamforming aberrations are present. Similarly, since the attenuation of ultrasound increases with frequency, at a given depth a lower transmit frequency must be used to produce a useable blood signal, and this reduces the resolution of the displayed blood velocity or power information.

It is generally accepted that the use of point-like reflectors greatly improves the accuracy and reliability of time-delay estimation. Unfortunately point-like reflectors are generally not available in ultrasound imaging of living tissue. A method of increasing the contribution of the blood component in signals would allow small blood vessels in certain tissue structures to be used as approximate point-like reflectors for time-delay estimation.

Therefore there is a need for an ultrasound imaging system with improved time-delay correction capabilities.

### BRIEF DESCRIPTION

Briefly in accordance with one aspect of the technique a method is provided. The method provides for extracting a blood component from an element signal produced by an element to obtain a filtered element signal, and extracting a blood component from a beamsum signal to obtain a filtered beamsum signal, calculating a time delay estimate between the filtered element signal and the filtered beamsum signal, or between the filtered element signal and the beamsum signal, or between the element signal and the filtered beamsum signal and applying the time delay estimate to correct transmit and receive beamforming time delays for the element. Systems and apparatus that afford functionality of the type defined by this method may be provided by the present technique.

In accordance with a further aspect of the present technique an ultrasound system is provided. The ultrasound system includes a transducer array comprising a set of array elements, each of the elements being separately operable to produce a pulse of ultrasound energy during a transmission mode and to produce an echo signal in response to energy reflected from an imaging object during receive mode, a transmitter coupled to the transducer array and being operable during the transmission mode to apply a separate transmit signal pulse with a respective time delay to each of the array elements such that a directed transmit beam is produced; a receiver coupled to the transducer array and being operable during the receive mode to sample the echo signal produced by each of the array elements as the vibratory energy impinges the imaging object and to impose a separate respective receiver time delay on each said echo signal sample to generate a corresponding plurality of receive signals, a clutter filter processor for filtering element signals to obtain a corresponding one or more filtered element signals and for filtering beamsum signals to obtain one or more filtered beamsum signals. The system further comprises a beamformer processor comprising a correlator processor for comparing the one or more filtered element signals with the one or more filtered beamsum signals, or the one or more filtered element signals with the beamsum signals or the element signals with the one or more filtered beamsum signals and a correlation sum processor for further processing to produce the beamforming time delays.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an ultrasound system implemented according to one aspect of the present technique;

FIG. 2 is a graphical representation of the signals in the clutter filtering according to one aspect of the present technique;

FIG. 3 is a block diagram of a clutter filter according to one aspect of the present technique;

FIG. 4 is a block diagram of clutter filter processing of the beamsum signal according to one aspect of the technique;

FIG. 5 is a block diagram of an example for calculating time delay estimates according to one aspect of the technique;

FIG. 6 is a schematic representation of an exemplary image display frame as obtained from one aspect of the technique;

FIG. 7 is a schematic representation of an exemplary image display frame as obtained according to another aspect of the technique; and

FIG. 8 is flow chart illustrating one method by which relative time delay estimates can be calculated in the ultrasound system.

### DETAILED DESCRIPTION

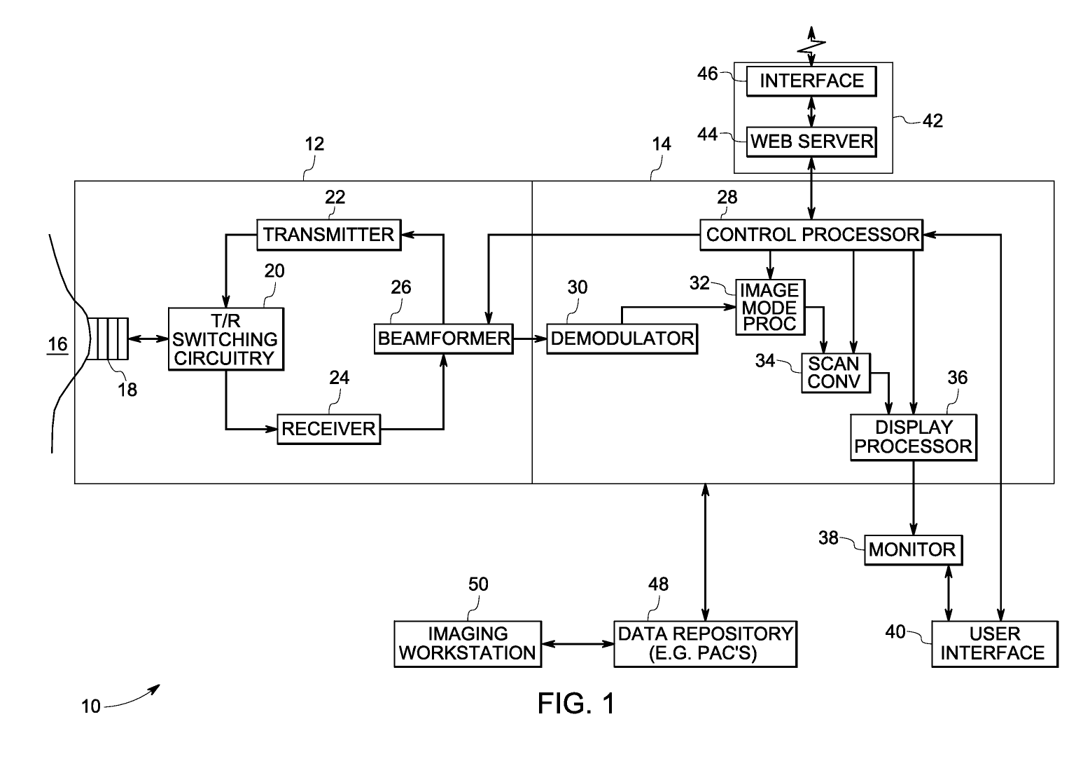

Embodiments of the present invention are generally directed to improving image resolution and contrast in an ultrasound system by calculating relative time delays of blood components in ultrasound signals. The embodiments of the present invention are generally directed towards cardiac imaging but may be used for imaging other areas such as, but not limited to, the abdomen. Referring now to FIG. 1, a schematic diagram of an exemplary ultrasound system 10 is illustrated in accordance with aspects of the present technique. The ultrasound system 10 includes an acquisition subsystem 12 and a processing subsystem 14. The acquisition subsystem 12 transmits ultrasound signals into a subject 16 and receives backscattered ultrasound signals from the subject 16. The acquired ultrasound signals are then processed by the processing subsystem 14 to generate an image of the subject 16.

The acquisition subsystem 12 includes a transducer assembly 18, typically an acoustic transducer assembly, which is in contact with a patient or subject 16 during an imaging procedure. The transducer assembly 18 may comprise a plurality of transducer array elements fabricated from materials, such as, but not limited to, lead zirconate titanate (PZT), polyvinylidene difluoride (PVDF) and composite PZT. It should be noted that the transducer assembly 18 is a two-way transducer and is configured to transmit ultrasound waves into and receive reflected ultrasound waves from the subject 16. In transmission mode, the transducer array elements convert the electrical energy into ultrasound waves and transmit it into the subject 16. In reception mode, the transducer array elements convert the ultrasound energy received from the subject (backscattered waves) into electrical signals.

In the illustrated embodiment, the acquisition subsystem 12 further includes transmit/receive switching circuitry 20, a transmitter 22, a receiver 24, and a beamformer 26. Although not illustrated, in one embodiment, a clutter filter processor may form an integral part of the beamformer 26. In another embodiment, the clutter filter processor may exist as a separate entity outside the beamformer 26. In such an embodiment, the clutter filter processor may be in operative association with the beamformer 26. The transmit/receive (T/R) switching circuitry 20 is coupled to the transducer array 18 for switching the transducer array 18 into transmission or reception mode. To generate ultrasound waves for transmission into the subject 16, the processing subsystem 14 sends transmit command data to the beamformer 26. In response to receiving the transmit command data, the beamformer 26 generates transmit parameters to create a beam of a desired shape originating from a certain point at the surface of the transducer array 18 at a desired steering angle. The beamformer 26 then sends the transmit parameters to the transmitter 22. The transmitter 22 uses the transmit parameters to encode transmit signals to be sent to the transducer array 18 through the T/R switching circuitry 20. The transmit signals are set at certain levels and phases with respect to each other and are provided to individual transducer elements of the transducer assembly 18. The transmit signals excite the transducer elements to emit ultrasound waves with the same phase and level relationships. As a result, a beam of ultrasound energy is formed in a subject 16 within a scan plane along a scan line when the transducer assembly 18 is acoustically coupled to the subject 16. This process is typically known as electronic scanning.

The transmitted ultrasound waves are then backscattered off the tissue and blood samples within the subject 16. The transducer array elements receive the backscattered waves at different times depending on the distance into the tissue they return from and the angle with respect to the surface of the transducer assembly 18 at which they return. As mentioned above, the transducer array elements receive the backscattered ultrasound signals from the subject 16 and convert these backscattered signals into electrical signals. Subsequently, the electrical signals are routed through the T/R switching circuitry 20 to the receiver 24. The receiver 24 amplifies and digitizes the received signals and may provide other functions such as gain compensation. The digitized received signals corresponding to the backscattered ultrasound waves received by each transducer element at various times preserve the amplitude and phase information of the backscattered waves. The digitized signals are then sent to the processing subsystem 14 through beamformer 26. The processing subsystem 14 sends receive command data to beamformer 26. The beamformer 26 uses the receive command data to form a receive beam originating from a point on the surface of the transducer assembly 18 at a steering angle typically corresponding to the point and steering angle of the previous ultrasound beam transmitted along a scan line. The beamformer 26 operates on the appropriate received signals by performing time delaying and focusing, according to the instructions of the command data from the control processor 28, to create received beam signals corresponding to sample volumes along a scan line in the scan plane within the subject 16. The phase, amplitude, and timing information of the received signals from the various transducer elements are used to create the received beam signals.

The processing subsystem 14 includes a control processor 28, a demodulator 30, an imaging mode processor 32, a scan converter 34 and a display processor 36. The control processor 28 interfaces with the imaging mode processor 32, the scan converter 34 and the display processor 36. Additionally the control processor 28 is responsible for sending transmit and receive command data to the beamformer 26. The demodulator 30 demodulates the received beam signals to create pairs of I and Q demodulated data values corresponding to sample volumes within the scan plane. In one embodiment, demodulation may be accomplished by comparing the phase and amplitude of the received beam signals to a reference frequency. The I and Q demodulated data values preserve the phase and amplitude information of the received signals.

The demodulated data is transferred to the imaging mode processor 32. The imaging mode processor 32 uses parameter estimation techniques to generate imaging parameter values from the demodulated data in scan sequence format. The imaging parameters may include parameters corresponding to various possible imaging modes such as B-mode, color velocity mode, spectral Doppler mode, and tissue velocity imaging mode, for example. The imaging parameter values are passed to the scan converter 34. The scan converter 34 processes the parameter data by performing a translation from scan sequence format to display format. The translation includes performing interpolation operations on the parameter data to create display pixel data in the display format.

The scan converted pixel data is sent to the display processor 36 to perform any additional spatial or temporal filtering of the scan converted pixel data, to apply grayscale or color to the scan converted pixel data, and to convert the digital pixel data to analog data for display on the monitor 38. The user interface 40 is coupled to the control processor 28 to allow a user to interface with the ultrasound system 10 based on the data displayed on the monitor 38.

The display processor 36 is further coupled to a display monitor 38 for displaying images. User interface 40 interacts with the control processor 28 and the display monitor 38. The control processor 28 may also be coupled to a remote connectivity subsystem 42 including a web server 44 and a remote connectivity interface 46. The processing subsystem 14 may be further coupled to a data repository 48 configured to receive ultrasound image data. The data repository 48 interacts with image workstation 50.

The components described in association with FIG. 1 may be dedicated hardware elements such as circuit boards with digital signal processors or may be software running on a general-purpose computer or processor such as a commercial, off-the-shelf personal computer, or specialized workstation. The various components may be combined or separated according to various embodiments of the invention. Thus, those skilled in the art will appreciate that the ultrasound system 10 described above is provided by way of example, and the present techniques are in no way limited by the specific system configuration.

In certain embodiments, color flow processing may be utilized to enhance the fast moving components of an ultrasound signal and suppress the slow moving components in such a signal. The fast moving components in the ultrasound signal may be representative of the blood component and the slow moving components in the signal may be representative of the tissue component. In these embodiments, a set of transmit firings may be made in a common imaging direction and the received element signals or the beamsum signals for each firing may then be stored in a memory. The set of firings are known as a set of “clutter firings.” The memory locations may be organized as a two-dimensional memory that stores a range sample number and a firing number. The element signals or the beamsum signals may be filtered in the firing number dimension, which is often referred to as filtering in “slow time.” The element signals or the beamsum signals may be filtered in the range dimension, which is often referred to as filtering in “fast time”. As will be appreciated, the high pass filtering in “slow time” reduces the tissue contribution at a range while preserving the blood component for that range. Further, filtering in “fast time” may modify the spectral content of a signal to improve its signal-to-noise ratio or to minimize aliasing artifacts prior to decimating that signal.

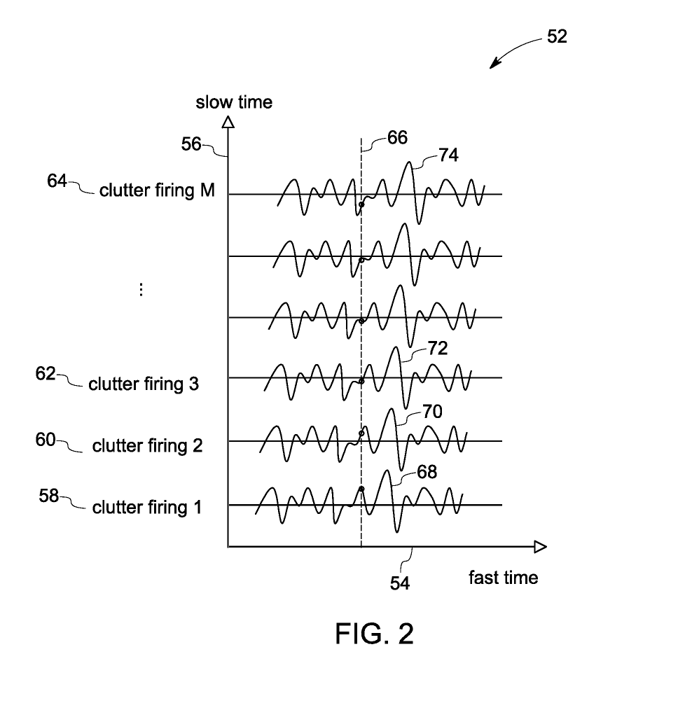

FIG. 2 is a graphical representation 52 illustrating fast time and slow time. The horizontal axis 54 represents the “fast time” or range dimension and the vertical axis 56 represents the “slow time” or firing number dimension. Signals 68, 70, 72 and 74 represent a set of received signals for M clutter firings 58, 60, 62 and 64 for the case in which there is a large fast moving component. The filled circles highlighted by dashed line 66 represent the clutter firing samples in “slow time” corresponding to a particular range. As will be appreciated, high pass filtering these samples in “slow time” will tend to suppress the slow moving component of the clutter firing signals at that range while tending to preserve the fast moving component. Repeating this high pass filtering in “slow time” at every range results in a set of M clutter-filtered signals.

In the implementation described above, a set of M filtered signals is obtained for each element in the transducer for each transmit firing direction. A corresponding set of M filtered beamsum signals may be obtained by applying the same clutter filtering to the sum of unfiltered element signals. Alternatively, in another embodiment, the corresponding set of M filtered beamsum signals may be obtained by summing the filtered element signals. A relative time delay may then be calculated between the filtered element signals and the corresponding filtered beamsum signals for each element and each of the transmit firing directions. Alternatively, a relative time delay may be calculated between the filtered element signals and the beamsum signals or the element signals and the filtered beamsum signals. The resulting time delay estimates for a given element and transmit firing direction may be combined, for example by averaging the time delays or calculating the median of the time delays, to produce a time delay estimate for that particular element and the corresponding firing direction. The variation in the estimates for a given element and firing direction may be used as an estimate of the reliability of the time delay estimate, so that only reliable time delay estimates may be used to modify the beamforming time delays. Additionally, as described in U.S. Patent Application US 2007/0167802(A1) the estimates for all elements for a given firing direction may be processed collectively to improve their reliability.

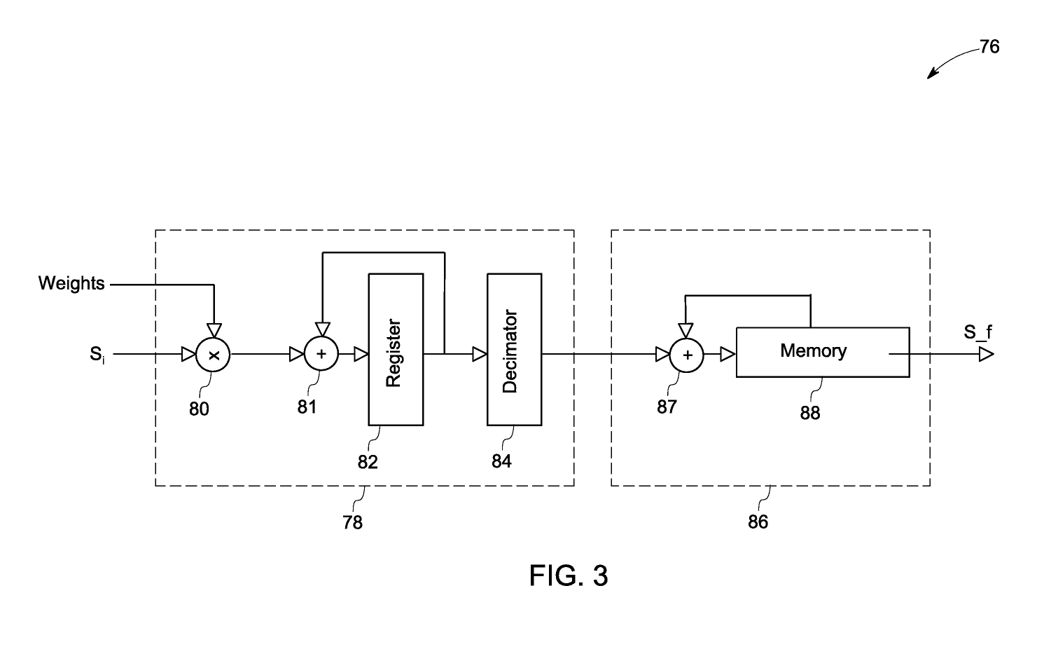

In a conventional color flow processing, as many as 16 or more clutter firings may be used to achieve a desired “slow time” filter response. The amount of memory required to store firing information typically increases with the number of clutter firings. As described in detail with regard to FIG. 3, the amount of memory required may be reduced by decimating the input signals before they are stored in memory and by calculating only one of the set of M clutter-filtered signals. Decimation can considerably reduce the cost and size of the hardware required. The reduction in memory may be particularly useful in the clutter processing of element signals since 256 or more element signals may be required to adequately sample the spatial variation of time delays due to aberrations in a human body. Therefore, a small clutter filter implementation makes it economically feasible to include the clutter processing in the same integrated circuit that implements beamforming time delay processing. Further, for a fixed number of memory locations, the accuracy and robustness of the time delay estimation may be improved by decimating the samples in range before storing them in the memory. The decimation filter can also be chosen to improve the signal-to-noise ratio of the signal when the sampling rate is much higher than twice the signal bandwidth. For example, in a cardiac ultrasound system, the typical signal bandwidth of interest may extend from zero frequency to about 5 MHz, while the sampling rate may be about 50 MHz. The weights may be chosen according to the well-known techniques for the design of finite impulse response (FIR) filters. According to the present embodiment, integer value weights may be chosen to reduce the size and cost of hardware.

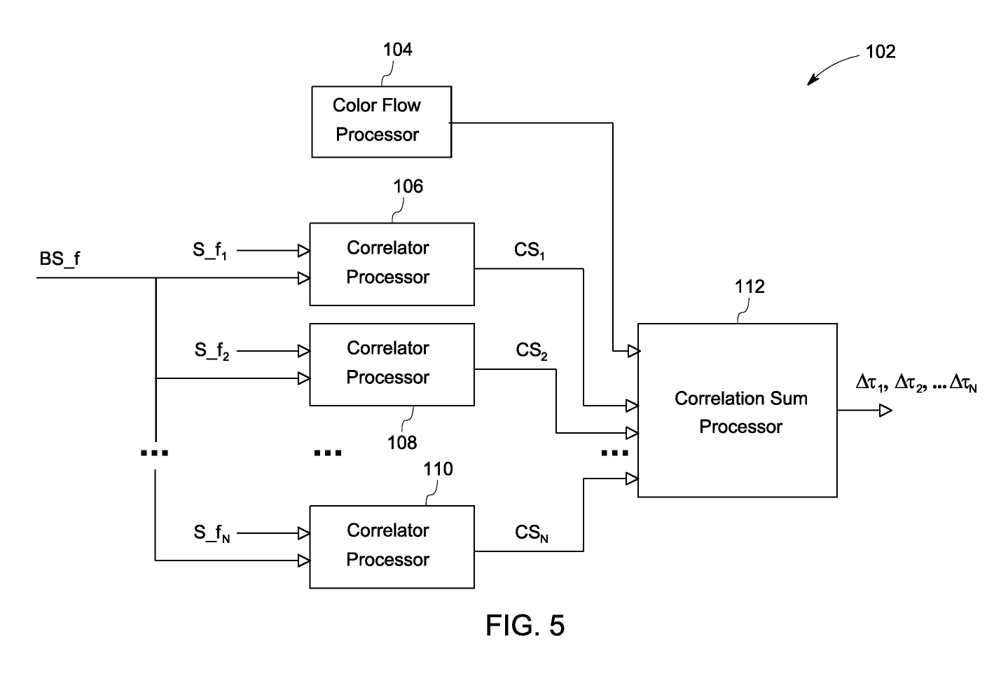

FIG. 3 is a schematic diagram of a clutter filter 76 according to another implementation of the present technique. In the illustrated embodiment, the clutter filter 76 includes a decimation block 78 and a clutter filter accumulator 86 coupled together. The decimation block 78 may further include a memory such as a register 82 and a decimator 84. The clutter filter accumulator may further include a memory 88. In the illustrated embodiment, the clutter filter processing may include filtering in range or “fast time” and decimation. As illustrated, a set of input signals Sᵢ (where i=1, 2, 3 . . . M) is multiplied by a set of weights in a multiplier 80, and a contiguous group, commonly known as a “block”, of the resulting samples are summed in the register 82. Thereafter, the decimator 84 sends the weighted sum of input signals in the block to the clutter filter accumulator 86. This process repeats for each block of samples in the input signal, thereby producing an output signal with fewer samples than the input signal. As illustrated, the clutter filter accumulator 86 comprises a memory 88 where the decimated signals may be accumulated and stored. The size of the memory 88 in the clutter filter accumulator 86 may be selected to accommodate the maximum desired number of decimated samples chosen by the operator or automatically by the system to contribute to time delay estimation. On the first clutter firing, the set of decimated samples from which a time delay estimate is to be produced may be written into the memory 88. On subsequent clutter firings, each decimated sample may be added via an adder 87 to the corresponding range sample stored in the memory 88. After the decimated samples from all the clutter firings have been added, the memory contains a filtered signal (S_f) with an enhanced blood component. This filtered element signal S_f may then be employed to calculate the relative time delays as described with regard to FIG. 5.

In one embodiment, the process as illustrated in FIG. 3 may be performed using a single multiplier 80, with the proper choice of weights, for the operations of filtering in “fast time” and filtering in “slow time”, thus reducing the size and cost of the implementation in hardware or the computational complexity of the implementation in software. In one example, three clutter firings with clutter weights [w1, w2, w3] and decimation by a factor of four using decimation filter weights [u1, u2, u3, u4] may be implemented. On the first clutter firing the weights [w1×u1, w1×u2, w1×u3, w1×u4] would be applied to each block of four samples. On the second clutter firing, the weights [w2×u1, w2×u2, w2×u3, w2×u4] would be used and the third clutter firing would use [w3×u1, w3×u2, w3×u3, w3×u4].

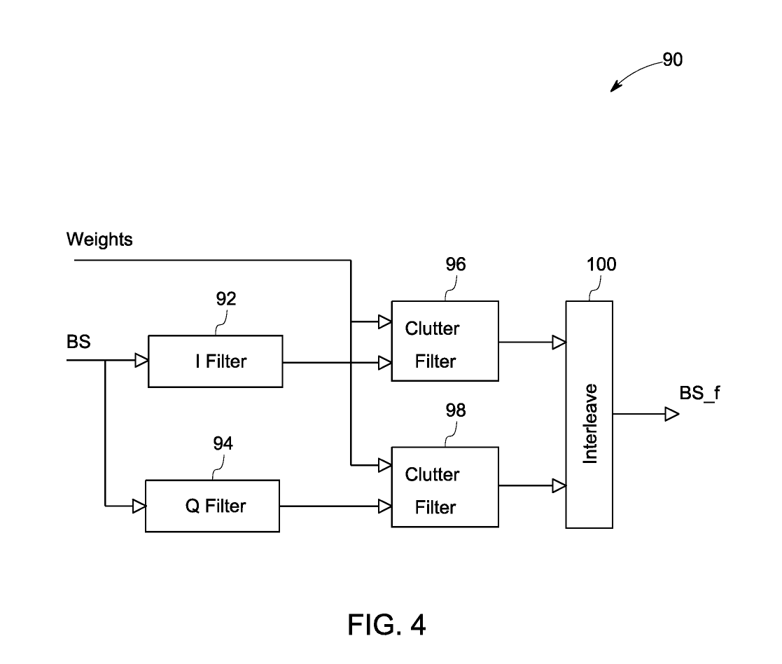

As will be appreciated, a beamsum signal is the weighted sum of two or more element signals. In the discussion that follows, the weights are assumed to be unity for simplicity. A filtered beamsum signal may be obtained by summing the filtered element signals as obtained in FIG. 3. Alternately, a filtered beamsum signal may be obtained by summing unfiltered element signals and then filtering the resulting sum. As described in U.S. Patent Application US2006/0004287(A1), one method of estimating beamforming time delay corrections converts the beamsum signal to a complex signal. This method is chosen as an illustration only. Other methods of estimating the time delay between signals known to those skilled in the art, such as methods which utilize two real signals or methods which utilize two complex signals, are equally applicable to the invention. FIG. 4 illustrates the process 90 by which the real beamsum signal BS is converted into a complex, clutter-filtered signal BS_f. As illustrated, an input real beamsum signal BS is converted to a bandpass, analytic signal form by applying a pair of filters 92 and 94. The real and imaginary parts of the analytic signal form, and the combined decimation- and clutter-filter weights may then be sent to a pair of clutter filters 96 and 98. The outputs from the pair of clutter filters 96 and 98 are the real and imaginary parts of the filtered beamsum signal BS_f. In the illustrated embodiment, the real and imaginary parts of the filtered beamsum signal are interleaved in block 100, as a single complex signal BS_f. This complex signal is the clutter filtered beamsum signal BS_f in which the blood component has been enhanced and tissue component has been suppressed.

FIG. 5 illustrates a process 102 to produce beamforming time-delay corrections. In the illustrated embodiment, each of the clutter filtered element signals, s_f₁, s_f₂, . . . , s_f_{N}, as obtained in FIG. 3 are compared with a reference beamsum signal BS_f as obtained in FIG. 4 in correlator processors 106, 108 and 110. The outputs of the correlator processors 106, 108 and 110 are the complex numbers CS₁, CS₂, . . . , CS_{N}, where the phase of each of the complex numbers is proportional to a time delay between the filtered element signal and the filtered beamsum signal. These complex numbers CS₁, CS₂, . . . , CS_{N} are further processed in the correlation sum processor 112, by means that includes but is not limited to smoothing, filtering and masking, to produce the beamforming time-delay corrections, Δτ₁, Δτ₂, . . . Δτ_{N}. The output of a color flow processor 104, which is available on many ultrasound imaging systems, operating on the beamsum data, may be an input to the correlation sum processor 112. The time delay corrections Δτ₁, Δτ₂, . . . Δτ_{N} may then be used to modify the transmit and receive beamforming time delays.

It will be appreciated that the illustrated embodiment represents just one illustrative example of the invention. Suitable modifications may be made by those skilled in the art. In one example, the element signals in the imaging system may be converted to a complex analytic signal or to a complex baseband form. In another example, the clutter filtering operations may be performed using a software processor rather than integrated circuits. Various methods of estimating the relative time delay between two signals are known to those skilled in the art and are equally applicable to the invention.

In one embodiment, a color flow processor 104 produces an estimate of the velocity and magnitude, or power, of a blood signal. In some embodiments, the estimate of the magnitude of the blood signal may be used by the ultrasound system to overlay blood flow velocity or power information on the image display for those pixels for which the power estimate is above a threshold value. Where the power estimate is below the threshold value, the blood flow velocity or power information may not be displayed. Further, the power flow estimate from the color flow processor 104 may be used as an additional input to the time delay estimation and may be used to identify regions of the image that contain significant blood flow. This additional input may be used by the system to automatically choose regions containing a significant blood component for processing by the time-delay estimation algorithm. In general, these regions may be irregular and non-contiguous in range and in beam direction. For example, the region chosen need not correspond to the same set of ranges for all beam directions. As another example, the region need not include all the beam directions. In regions for which the color flow processor output indicates that there is little blood component, time-delay estimation can be performed in the conventional manner, without using clutter filtering, so that time delays are estimated using the tissue component of the signal. In this way, reliable time-delay estimates can be obtained for regions both with and without significant blood components.

In one embodiment a contrast agent may be used to increase the size of the blood component in the beamsum signal, or the element signal, or both the beamsum signal and the element signal. The injection of contrast agents in the bloodstream may be used to increase the reliability and robustness of the time delay estimates by increasing the amplitude of the blood component with respect to the tissue component.

In certain embodiments, the displayed output from the color flow processor may be used by an operator to choose regions on the image display for time delay estimation.

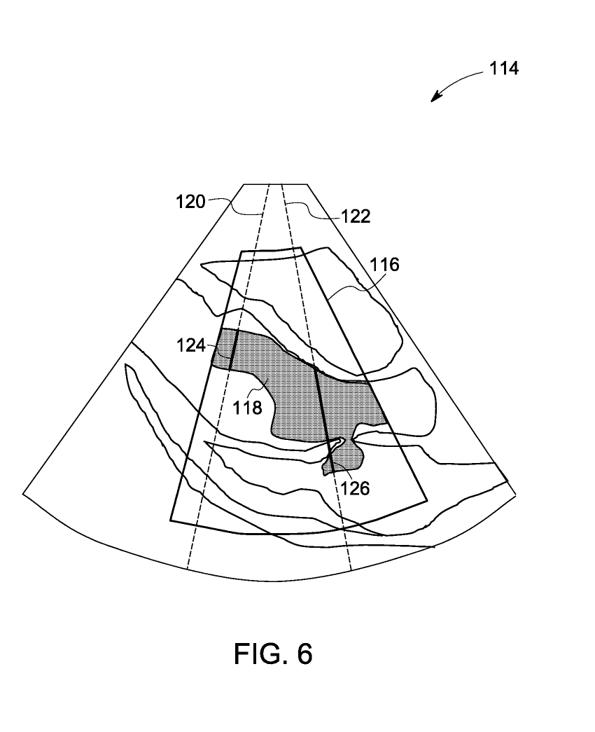

FIG. 6 illustrates one example of using the output of a color flow processor 104 to improve the accuracy and reliability of time-delay estimation using clutter filtering. As illustrated, significant blood flow is detected by the color flow processor in region 118, which lies in region of interest 116 on image display 114. For beam direction 120, the set of samples 124 have been identified as a contiguous set for which a substantial fraction of the samples in the contiguous set contain a significant blood component. These samples will be input to the clutter filter time delay estimation blocks. Similarly, the set of samples 126 for beam direction 122 have been identified.

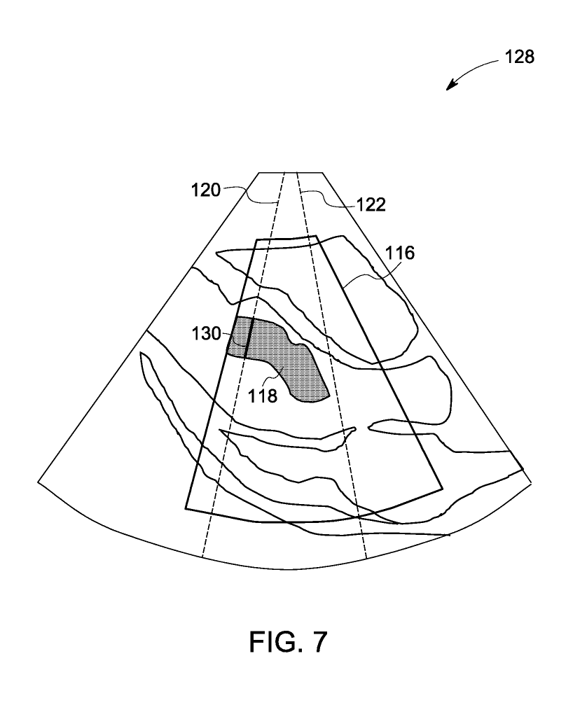

FIG. 7 illustrates the image display frame 128 a short time after the image display frame 114, illustrated in FIG. 6. The figure illustrates a new set of range samples 130 for time delay estimation that is calculated for beam direction 120 using the new output from the color flow processor 104. As illustrated, beam direction 122 does not have significant blood flow for this frame, therefore the clutter-filter time delay estimation for this beam direction is not used.

In FIG. 6 and FIG. 7, a contiguous set of samples are chosen for each beam, but in other embodiments non-contiguous sets can be used. More generally, the output of the color flow processor 104 can be used to weight the contribution of each sample in the image to the clutter filter time-delay estimation.

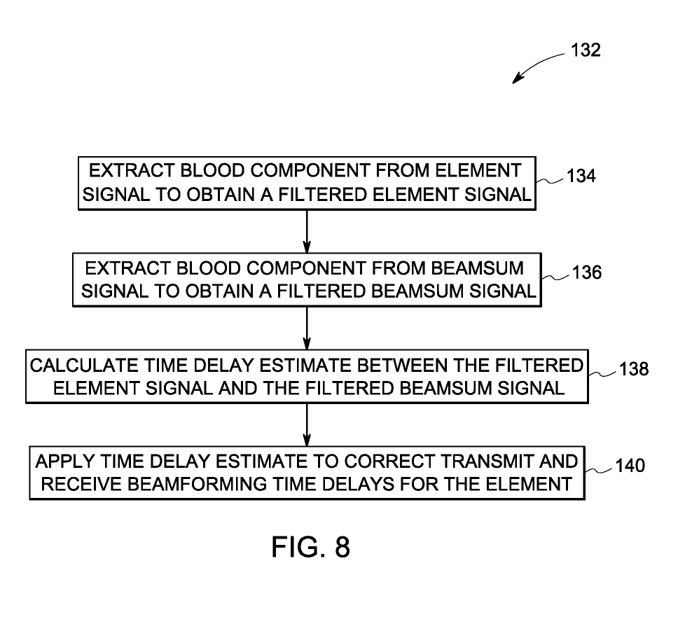

FIG. 8. is a flow chart illustrating a control scheme 132 for time delay estimation to correct transmit and receive beamforming time delays in ultrasound signals according to one embodiment of the present technique. In the illustrated embodiment, the control scheme 132, includes the steps of extracting a blood component from an element signal to obtain a filtered element signal at step 134, extracting a blood component from a beamsum signal to obtain a filtered beamsum signal at step 136, calculating a time delay estimate between the filtered element signal and the filtered beamsum signal at step 138. Further, the control scheme 132 includes the step of applying the time delay estimate to correct the transmit and receive beamforming time delays for the element at step 140.

The above discussed techniques of enhancing ultrasound image resolution and contrast have many advantages including improved cardiac image resolution and improved color flow sensitivity. Moreover, the technique also provides for improved image resolution for difficult subjects such as abdominal imaging of heavy patients as well as for breast imaging.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method comprising:
extracting a blood component from an element signal produced by an element to obtain a filtered element signal, and extracting a blood component from a beamsum signal to obtain a filtered beamsum signal;
calculating a time delay estimate between the filtered element signal and the filtered beamsum signal, or between the filtered element signal and the beamsum signal, or between the element signal and the filtered beamsum signal; and
applying the time delay estimate to correct transmit and receive beamforming time delays for the element.

2. The method of claim 1, further comprising:
converting a real beamsum signal into a complex beamsum signal.

3. The method of claim 1, wherein extracting the blood component from the element signal or the beamsum signal comprises filtering and decimating the element signal or the beamsum signal.

4. The method of claim 1, further comprising employing a contrast agent to increase a size of the blood component in the beamsum signal, or the element signal, or both the beamsum signal and the element signal.

5. The method of claim 1, wherein extracting the blood component comprises extracting the blood component from a region in which a size of the blood component is significantly less than a size of a tissue component.

6. The method of claim 1, wherein calculating the time delay estimate comprises comparing one or more filtered element signals to one or more filtered beamsum signal in a correlator processor.

7. The method of claim 1, wherein extracting the blood component comprises suppressing slow moving components of the element signal and enhancing fast moving components of the element signal or suppressing the slow moving components of the beam sum signal and enhancing the fast moving components of the beamsum signal.

8. The method of claim 7 further comprising applying color flow processing to suppress the slow moving components of the element signal and to enhance the fast moving components of the element signal or to suppress the slow moving components of the beamsum signal and enhance the fast moving components of the beamsum signal.

9. The method of claim 7, wherein calculating the time delay estimate comprises employing a power estimate from color flow processing as an input.

10. The method of claim 9 wherein the color flow processing identifies regions of signals that have a blood component.

11. The method of claim 9, wherein a sample in the element signal or the beamsum signal that contains more blood component contributes more to a time delay estimation.

12. The method of claim 1, comprising calculating the time delay estimate on regions that contain blood flow.

13. The method of claim 1 further comprising:
applying a clutter filter to multiple acquisitions of the element signal to obtain one or more filtered element signals;
applying a clutter filter to multiple acquisitions of the beamsum signal to obtain one or more filtered beamsum signals; and
calculating a time delay estimate between the one or more filtered element signals and the one or more filtered beamsum signals, or between the one or more filtered element signals and one or more beamsum signals, or between one or more element signals and the one or more filtered beamsum signals.

14. An ultrasound system comprising:
a transducer array comprising a set of array elements, each of the elements being separately operable to produce a pulse of ultrasound energy during a transmission mode and to produce an echo signal in response to energy reflected from an imaging object during receive mode;
a transmitter coupled to the transducer array and being operable during the transmission mode to apply a transmit signal pulse with a respective time delay to each of the array elements such that a directed transmit beam is produced;
a receiver coupled to the transducer array and being operable during the receive mode to sample the echo signal produced by each of the array elements and to impose a separate respective receiver time delay on each said echo signal sample to generate a corresponding plurality of receive signals;
a clutter filter processor for filtering element signals to obtain a corresponding one or more filtered element signals and for filtering beamsum signals to obtain one or more filtered beamsum signals; and
a beamformer processor comprising
a correlator processor for comparing the one or more filtered element signals with the one or more filtered beamsum signals, or the one or more filtered element signals with the beamsum signals, or the element signals with the one or more filtered beamsum signal; and
a correlation sum processor to produce the beamforming time delays.

15. The ultrasound system of claim 15, further comprising a color flow processor to identify regions in the imaging object that contain blood flow.

16. The ultrasound system of claim 15, wherein the clutter filter processor includes a filter and a decimator.

17. The ultrasound system of claim 15, wherein the clutter filter processor includes a memory.

18. The ultrasound system of claim 15, wherein the clutter filter extracts a blood component in a region where the blood component is smaller than a tissue component.
